(19) United States
(12) Patent Application Publication
(10) Pub. No.: US 2024/0329023 A1
(43) Pub. Date: Oct. 3, 2024

(54) Detection Case for Portable Device

(71) Applicant: Porsentech ApS, Pandrup (DK)

(72) Inventor: Kathrine Marie ELMER, Blokhus (DK)

(21) Appl. No.: 18/740,122

(22) Filed: Jun. 11, 2024

Related U.S. Application Data
(63) Continuation of application No. PCT/DK2022/050277, filed on Dec. 13, 2022.

(30) Foreign Application Priority Data
Dec. 13, 2021 (DK) PA 2021 70613

(51) Int. Cl. G01N 33/00; G01N 33/497; G08B 17/107; G08B 21/16; G08B 21/18; H02J 50/00; H04B 1/3888

(52) U.S. Cl. CPC G01N 33/0063; G01N 33/4972; G08B 17/107; G08B 21/16; G08B 21/182; H02J 50/001; H04B 1/3888

(57) ABSTRACT

A detection case that receives and fully or partially covers a portable device has a detection system for detecting one or more hazardous particles and/or gases, without interfering with the daily use of the portable device. A method for detecting smoke and/or gas when using a detection case, or when the detection case is located unattended next to a user, is also disclosed.

### CROSS-REFERENCE TO RELATED APPLICATIONS

This application is a continuation under 35 U.S.C. 111 of International Patent Application No. PCT/DK2022/050277, filed Dec. 13, 2022, which claims the benefit of and priority to Danish Application No. PA 2021 70613, filed Dec. 13, 2021, each of which is hereby incorporated by reference in its entirety.

### FIELD OF INVENTION

The present invention relates to a detection case for a portable device.

### BACKGROUND

Portable gas and/or smoke devices are well known in the industry as an industrial application. The portable gas and/or smoke devices have only one purpose, which is to detect gas and/or smoke in the area of the device or the worker who is carrying the device. This reduces the hazard risk during work for the worker. The portable gas and/or smoke devices may be a locally or a centrally controlled detection system.

The problem is that the devices are often provided for industrial applications for professional use, and not for private use, as for example in households, campers, on a boat, when traveling or when people are in the nightlife.

One situation which might occur in a private home, or in a camper, or on a boat, is the risk of fire, which may occur because of unconcentrated attention when smoking cigarettes or during use of candles etc.

Another situation which might occur in a private home, or in a camper, or on a boat, is gas hazard which may occur during cooking using gas. Furthermore, it is also well known that gas attacks in caravans occur. Thieves are pumping noxious gases into the caravans to make the families unconscious, so the thieves can rob them. The families are left traumatized after such an assault.

US 2019/221101 A1 discloses a carbon monoxide gas detection and warning system for a portable phone device which includes a phone case dimensioned and configured to receive a portable phone device, the phone case having a raised outer shell portion that includes at least one sensing port communicating with a corresponding sensing chamber formed within a wall of the shell, and a sensing circuit embedded in the shell of the phone case, the sensing circuit including a carbon monoxide sensor operatively associated with the sensing chamber of the at least one sensing port for generating a gas detection signal upon detecting the presence of carbon monoxide gas in the sensing chamber, and a controller for receiving the gas detection signal from the carbon monoxide sensor and for periodically sending an output signal to the portable phone device by way of a wireless transmitter.

US 2020/082701A1 discloses a monitoring system for monitoring an environment and generating data in response to the presence of at least one chemical hazardous to human health in the environment. The monitoring system comprises a detector component, which may be embedded in the shell of the phone case. The detector component comprises a plurality of sensors, wherein the sensors are installed in the case based on which certain chemicals must be detected. The monitoring system is configured to communicate the data to an associated personal communication device or the like for processing. The monitoring system can be in the form of a selectively attachable component.

US20140201182A1 discloses a system and a method for a mobile electronic system that gathers and analyzes odors, airborne chemicals and/or compounds. A signature or representation of the odors, airborne chemicals and/or compounds can be generated. Extrinsic data associated with the odors, airborne chemicals and/or compounds or capturing the odors, airborne chemicals and/or compounds can be identified. A model can be generated based on the representation and the extrinsic data. Filters can be generated based on the extrinsic data. The model can be searched for candidate matches, solutions, or other results based on the representation and the filters. Results can be generated based on the search and candidate matches. This solution is, however, not suitable for detecting hazardous particles and/or gases in everyday life situations, in which various gasses and/or particles from the surroundings constitute noise.

Most people are aware of the hazard that comes from smoke, fire, and gas, but nevertheless, a lot of people do not bother to install an alarm in their home and other related locations or walk around with a detection kit. There is a lack of consumer products, which are easy to handle for a private person. The products available on the market are very expensive, highly advanced, unhandy, and aimed at industrial use.

### BRIEF DESCRIPTION

It is an object of the present systems to provide a detection case for detecting hazardous particles and gases adaptable for a portable device, wherein the detection case does not interfere with the daily use of the portable device. The object is also to provide a method for detecting smoke and/or gas when using the detection case, or when the detection case is located unattended next to the user.

The present disclosure addresses these objects by providing a detection case for a portable device, the detection case configured to receive the portable device, wherein the detection case comprises a detection system configured to detect hazardous particles and/or gases, wherein the detection system is arranged adjacent to an inner surface of the detection case.

There is a need for a detection system, which is capable of detecting the presence of gases in a surrounding area of a user. Such a detection system is personal and must be portable, and capable of taking care of the user's safety. For example, a situation which might occur in nightlife, where people are exposed to alcohol and drugs. In this situation, it would be an advantage to measure and detect blood alcohol content, BAC, and/or date rape drug content.

The detection case comprises the detection system for detection of hazardous particles and/or gases. The hazardous particles may be smoke particles etc. The detection of hazards gases may be gases evaporating from chemical materials and/or compounds. The detection case is adaptable for a portable device. The detection case may fully or partially cover the portable device. The portable device may be a mobile phone, tablet, laptop or similar device. The detection case may be the housing case of a mobile phone. The detection case may be the housing case of a tablet or a laptop or a similar device. The portable device may comprise components for wireless communication with external detection and/or warning systems.

Portable devices are used widely, and often arranged in a case, such as a protection case. The detection system is comprised in the detection case. The detection system is arranged adjacent to an inner surface of the detection case. The user may easily install the portable device in the detection case, and easily activate the detection system. The detection case may also function as a protection case. The user of the portable device may therefore easily carry the detection case in places where the user is without any inconvenience to the user. The detection system can alert the user in a personal way.

The detection case is configured to enclose fully or partially the detection system. The detection case is configured to receive the portable device through an opening. The detection case may cover at least a part of the portable device.

The detection case for a portable device is personal, portable, convenient, and the detection system has a high reliability and safety level. The user may easily carry the detection case to different locations as an extra protection item for the portable device. The detection case will therefore always be within a short distance of the user.

Alternatively, a portable device comprises a detection system having detection components for detecting hazardous particles and/or gases, wherein the portable device is a portable communication device, such as a mobile phone, a tablet, a laptop, or a similar personal computer. The detection case may be a detection housing of a portable device, wherein said detection housing comprises a detection system configured to detect hazardous particles and/or gases, wherein the detection system is arranged adjacent to an inner surface of the detection housing.

The detection system, which is built in a detection case, may equally be built in a portable device as well. The detection system may be comprised in the housing of the portable device in such a way that the detection system is capable of detecting hazardous particles and/or gases. The detection system, which is related to the detection case or housing, may perform the detection substantially equally when built in the portable device. The detection system, which is built in a portable device also has a high reliability and safety level. The user may easily carry the portable device to different locations, and be provided with an extra protection feature. The portable device can easily be kept within a short distance of the user.

In an embodiment, the detection case comprises a protection plate for protecting the detection system, such that the detection system is arranged between said inner surface of the detection case and the protection plate.

The detection case may comprise a protection plate for protecting the detection system's electronic components and PCB, such that the detection system is arranged between the inner surface and the protection plate. Alternatively, the detection system is arranged adjacent to an inner surface of the detection case wherein the detection system may be molded or partly molded into the inner surface of the detection case, which will increase the protection of the detection system's electronic components and PCB. The molding will still place the detection system between an inner surface and a protection plate, but where the inner surface and the protection plate are molded together this encloses and protects the detection system.

The detection case has a limited thickness to ensure the detection case is easy to handle for a private person. The detection case provides a solution which is economically advantageous, highly advanced, handy, and aimed for personal use, but does not exclude industrial use.

The detection system's electronic components and PCB are arranged such that the detection system's electronic components and PCB are distributed evenly along the inner surface of the detection case. The length and width of the detection system may be less than the detection case volume for housing a mobile device. The height of the PCB may define the distance between the inner surface and the protection plate. Alternatively, the height of the PCB may define the distance between the inner surface and a back side of mobile device. The distance between the inner surface and the back side of mobile device is related to the distance when the mobile device is resting in the protection case, wherein the distance may be less than 10 mm. The height of the PCB may be less than 7 mm. The height of the PCB may even be less than 6 mm or less than 5 mm.

In an embodiment, the detection case comprises at least one aperture, wherein at least one detection unit is arranged adjacent to the inner surface of the detection case relative to the aperture, wherein the aperture is configured to provide a fluid communication between the ambient surrounding and the detection unit.

The detection case also comprises at least one detection cavity or aperture. The detection cavity comprises at least one aperture, such that the hazardous particles or gases are capable of at least entering the detection cavity or aperture. Also, liquid may be capable of entering the aperture, such that the liquid may be tested for hazardous particles. The detection cavity may be arranged adjacent to the inner side of the detection case. The detection cavity may be in fluid communication with the ambient surroundings. The detection components may be arranged on the inner side of the detection case, wherein the detection components are configured to detect the presence of hazardous particles and/or gases inside the detection cavity through an aperture in the detection case. The detection cavity may comprise detection components for detecting hazardous particles and/or gases.

The detection cavity may comprise at least one detection cavity, wherein the detection cavity comprises at least one aperture, such that the hazardous particles are capable of entering the detection cavity, wherein the detection components are configured to detect the presence of hazardous particles inside the detection cavity. The detection components are configured to detect the presence of hazardous particles which are substantially near or in contact with the detection cavity. The detection components may comprise a material which interacts with the hazardous particles and/or gases, such that the presence of hazardous particles and/or gases, can be detected using the detection components. The detection components may include solid state detection units such as gas sensors or the like.

The detection cavity comprises at least one detection cavity, which may be provided with at least one aperture, such that the hazardous particles are capable of entering the detection cavity. The detection components are configured to detect the presence of hazardous particles and/or gases inside the detection cavity. The detection components may comprise sensing material, IR and/or an ion detector, for example.

Alternatively, a portable device comprises at least one detection cavity arranged in the housing of the portable device. The detection cavity may comprise at least one aperture. The hazardous particles and/or gases are capable of entering the detection components in the housing of the portable device.

The detection components may comprise at least one detection unit, wherein the detection unit is arranged adjacent to an inner surface of the detection case. At least one detection cavity may be arranged inside the detection case. The detection cavity may be arranged inside the detection case, such that the detection cavity is adjacent to an inner surface of the detection case. The detection case may comprise at least one aperture in communication with the detection cavity. The detection cavity may be comprised in the detection unit. The detection cavity may be arranged relative to the detection unit.

In an embodiment, the detection case comprises at least one protrusion member on an outer surface, such that when the detection case is arranged on a resting surface with the outer surface facing the resting surface, the protrusion member provides space between the outer surface and the resting surface.

The detection case may comprise at least one protrusion on an outer surface of the detection case. The protrusion or protrusions may be arranged along the case edge facing towards a resting surface. The detection case is arranged on a resting surface with the outer surface facing the resting surface, such that the protrusion provides space between the outer surface and the resting surface. The space between the outer surface and the resting surface allows the hazardous particles and/or gases to flow along the outer surface of the detection case and flow in through the aperture such that the detection system is capable of detecting the presence of hazardous particles and/or gases.

In an embodiment, the detection unit is configured to detect more than one type of hazardous particle and/or gas.

The detection system may comprise a plurality of detection units. The detection system is configured to detect more than one type of hazardous particle and/or gas. The detection system may comprise a detection unit, which is configured to detect more than one type of hazardous particle and/or gas. The detection system may detect one or more of the following hazardous particles and/or gases, such as the contents of smoke, pollution, air, liquid and/or radiation. A detection system may, for example, measure:
Smoke/fire detection.
Alcohol and/or drugs in drinks.
Alcohol percent in liquid.
Blood alcohol concentration, e.g. breathalyzer.
Air quality, e.g. contents of pollution
Ambient temperature
CO, CO₂, CH₄, H₂S, C₄H₁₀, C₃H₈, CH₃CH₂OH, NH₃
C₂H₃Cl₃, CH₂CCl₃, CF₃CH₂F, CHCl₃, CHClF₂, C₂H₆O, C₄H₁₀, CH₃CH₃, C₂H₆.

The detection unit may be an intelligent sensor, which is configured to measure for example temperature, barometric pressure, humidity, air quality, gas sensing and distinguish different gas compositions. The detection unit may detect gases by measuring their unique electronic fingerprint and therefore distinguish different gas compositions. The detection unit may be a sensor comprising machine learning, ML. Alternatively, Artificial Intelligence, AI, features detect predetermined hazardous particles and/or gases. The detection system may allow for programming of predetermined customized settings of measuring parameters. The detection unit may detect Volatile Organic Compounds, VOCs, volatile sulphur compounds, VSCs, and other gases such as carbon monoxide and hydrogen in the part per billion, ppb, range. The detection of hazardous gases may be gases evaporating from chemical materials and/or compounds.

The detection unit may comprise a gas scanner function. The presence of VSCs may be detected as an indicator for e.g. bacteria growth. The gas scanner may be customized with respect to sensitivity, selectivity, data rate and power consumption. The detection unit and/or the detection system may be trained to a specific customized application with the use of machine learning, ML, or alternatively Artificial Intelligence, AI, features. An application on an external system may also be trained to a specific customized application with the use of machine learning, ML, or alternatively Artificial Intelligence, AI, features.

In an embodiment, the detection system comprises a power storage unit, wherein the power storage unit is configured to receive power from a charging unit.

The detection system may comprise a power storage unit for providing power to the detection system. The power storage unit is configured to receive power from an external charging unit. The power storage unit may comprise one or more batteries or the like.

In an embodiment, the charging unit comprises a wireless charging device configured to receive wireless charging.

The detection system may comprise a power storage unit, wherein the power storage unit is configured to receive power from a wireless charging unit. A portable device may for example be charged using a wireless charging phone case. The detection system may be wirelessly charged at the same time as the portable device, for example when using a wireless charging phone case. The detection system may comprise a magnetic part which may be used when placing the detection case on a wireless charging magnetic phone case.

In an embodiment, the charging device comprises a coil.

The wireless charging device may comprise a coil for harvesting power from a wireless charging mobile device case. The wireless charging device may comprise a coil for harvesting power from, for example, a wireless charging magnetic phone case.

In an embodiment, the detection system comprises transmitting components configured to send at least one warning signal, such that when the detection system detects the hazardous particles and/or gases, the detection system transmits at least one warning signal, such that a user becomes aware of the hazardous situation.

The detection system may comprise transmitting components for transmitting one or more warning signals when detecting hazardous particles and/or gases. The detection system may transmit the warning signals to the portable device. The detection system may detect hazardous particles and/or gases using the detection components. The detection system comprises transmitting components, such that when the detection system detects the hazardous particles and/or gases, the detection system is capable of sending at least one warning signal to the portable device.

The transmitter may be configured to transmit a warning signal to a sound emitting unit. The sound emitting unit may be a buzzer unit or a beeper unit. The buzzer/beeper unit may be comprised in the detection case. A buzzer or beeper unit is configured to be activated by a control unit. The sound emitting unit may for example be a mechanical, an electromechanical, or a piezoelectric unit.

The transmitter may be configured to transmit a visual warning signal. The visual warning signal may be a material or component, which changes color when receiving the warning signal. For example, a solid-state component, such as a light emitting diode or the like.

The detection system may comprise transmitter for transmitting one or more warning and/or information signals. The detection system may transmit warning and/or information signals to the portable device. The detection system may detect hazardous particles and/or gases using the detection components. When the detection system detects hazardous particles and/or gases, the detection system sends at least one warning signal to indicate the presence of hazardous particles in the surroundings. Alternatively, when the detection system detects hazardous particles and/or gases, the detection system sends at least one warning signal to the portable device. When the detection system detects hazardous particles and/or gases, the detection system sends at least one information signal to the portable device, regarding for example content level.

Alternatively, the detection system comprises transceiver, such that when the detection system detects hazardous particles, the detection system may be capable of sending at least one warning signal to the portable device and receiving a reply signal from the portable device. The detection system may comprise transceiver for sending and receiving signals. When the detection system detects hazardous particles in the detection cavity, the detection system is capable of sending at least one warning signal to the portable device and, if configured to do so, also receiving reply signals from the portable device.

The detection system, which alternatively may be built in the portable device comprises transmitter. When the detection system detects hazardous particles and/or gases inside the detection cavity, the detection system is configured to send at least one warning signal, such that a user becomes aware of the hazardous situation.

The detection system comprises a control unit. The control unit may be configured to control the detection system for detecting hazardous particles and/or gases. The control unit may be configured to control the transmitter for transmitting signals to the portable device. Alternatively, the control unit may be configured to control the transceiver for transmitting signals to the portable device and receiving the signals sent from the portable device to the detection system. The control unit may be configured to control the wireless communication to and from the detection system.

In an embodiment, the detection system comprises a processor for processing at least one detected hazardous particle and/or gases, wherein the detection system is configured to identify at least one of the detected hazardous particles and/or a concentration level of the hazardous particles and/or gases.

The processor is configured to process the signals from the detection system according to at least one predetermined parameter or setting. The predetermined parameters or settings may comprise one or more detection levels, wherein the detection levels determine when the detection system transmits a warning signal to the portable device.

The detection system, which alternatively may be built in the portable device, comprises a control unit. The control unit may be configured to control the detection system for detecting hazardous particles and/or gases. The control unit may be configured to reprogram or reinstall data in the detection system, such that the detection system settings, such as type of particles and/or gases and/or hazardous level for detecting hazardous particles and/or gases can be changed.

In an embodiment, the detection system comprises a processor for processing a plurality of detected hazardous particles and/or gases simultaneously, wherein the detection system is configured to identify a plurality of the detected hazardous particles and/or a concentration level of the hazardous particles and/or gases.

The detection system comprises a processor for processing a plurality of detected hazardous particles and/or gases simultaneously. The detection system may be configured to control the detection components for detecting a plurality of hazardous particles and/or gases at the same time. The detection system may be configured to detect at least two different hazardous particles and/or gases simultaneously. Alternatively, the detection system may be configured to detect at least four different hazardous particles and/or gases simultaneously. Optionally, the detection system may be configured to detect a plurality of hazardous particles and/or gases simultaneously. The detection system may also transmit a warning signal which provides information related to the one or more detected particles and/or gases. The detection system or part of the detection system may be configured to be reprogrammable, such that the detection settings, such as type of particles and/or gases and/or hazardous level for detecting hazardous particles and/or gases can be changed.

In an embodiment, the detection system is configured to be in data communication with a portable device.

The detection system comprises communication components configured to communicate with a portable device or an external stationary device. The communication components are configured to handle data communication between the detection system and the portable device. The communication components may comprise a wireless communication unit. The wireless communication unit is configured to communicated with a portable device or an external stationary device. The wireless communication unit may also be configured to be in data communication with other portable devices, than the portable device arranged in the detection case.

In an embodiment, the detection system is configured to receive instructions from a portable device, such that the detection system is configured to detect predetermined hazardous particles and/or gases by a user using said portable device.

A user may use a portable device or an external computer to configure the detection system, such that the settings for the detection system for detecting hazardous particles and/or gases can be initialized or changed. The user may use a user interface to access the detection system or part of the detection system, such as the detection units. A control unit may be configured to control the components for wireless or for wired communication to and from the detection system. The control unit may be programmed using the portable device. The portable device may comprise an app solution from where a user may interact with the detection case though a user interface related to the portable device.

The detection case may comprise a computer-readable storage medium unit. The control unit may be in data communication with the computer-readable storage medium unit. Alternatively, the control unit may be in data communication with the computer-readable storage medium unit related to the portable device.

The control unit may store a log file and/or data in a log file. The log file may be stored on the computer-readable storage medium unit in the detection case. Alternatively, the log file may be stored on the computer-readable storage medium unit related to the portable device or related to a cloud solution. The log file may be accessed from an app solution. The app solution may be installed on the portable device. Alternatively, the app solution may be installed on other devices. The other devices may be in data communication with the computer-readable storage medium unit where the log file is stored.

The control unit is configured to analyze and process the measurements retrieved by the detection system inside the detection cavity or inside the detection unit. The control unit may be a part of the detection unit. The control unit uses a processor configured to analyze the contents of the hazardous particles and/or gases. The measurement(s) may, for example, be stored in the log file. The log file may be accessed from user interfaces related to other devices. The log file may comprise a track record related to measurements of hazardous particles, when measured over time.

The detection system may be built in a detection case which is configured to enclose or partially enclose a housing of a portable device. The detection system, which may be built in the housing of the portable device, comprises communication components configured to communicate with other portable devices. Alternatively, the detection system is configured to use the portable device's communication components. The portable device's communication components may be configured to communicate with other portable devices.

The detection case may have multiple sides. The detection case may for example be quadrangular, having four sides. For example, as a portable electronic device, such as a mobile phone, a tablet, or a laptop, may have four sides along the edges of the portable device. The detection cavity may be arranged in extension from a first side of the detection case.

The detection cavity may be arranged along a side or part of a side of the detection case. The detection cavity may be arranged along two sides or part of two sides of the detection case.

The housing of the portable device may have a predetermined shape, which defines the sides of the portable device. The portable device has multiple sides. The portable device may for example be quadrangular, having four sides. The portable device may be a mobile phone, a tablet, or a laptop, and may have four sides along the edges of the portable device. The detection cavity may be arranged in extension from a first side of the portable device. The detection cavity may be arranged along a side or part of a side of the portable device. The detection cavity may be arranged along two sides or part of two sides of the portable device.

The detection case comprises a detection cavity and a reference cavity. Alternatively, the detection case comprises a detection unit and a reference unit. The detection system may be configured to compare the measurement from the detection cavity to the measurement from the reference cavity or unit. If the value of the measurement from the detection cavity differs from the value of the measurement from the reference cavity, there may be hazardous particles and/or gases in the surroundings which may cause a hazard situation. The detection system may send a warning signal, such that the user will become aware of the hazardous situation.

The portable device may comprise a reference cavity or unit, such that the detection system is capable of comparing a measured, detected value related to the detection cavity to a measured reference value related to the reference cavity or unit. The reference cavity or unit may be arranged in a position, which extends from a side of the detection case. The reference cavity may be arranged in extension from a side of the detection case, different than the first side from which the detection cavity extends. Alternatively, the reference cavity may be arranged such that the reference cavity extends from the same side as the first side the detection cavity extends from. The reference cavity may be arranged parallel to the detection cavity. The reference cavity may be arranged along a side or part of a side of the detection case. The detection cavity may be arranged along two sides or part of two sides of the detection case. The portable device may comprise a reference cavity, wherein the reference cavity is arranged in extension from a second side of the detection case.

The detection system may be powered from at least one battery. The battery may be arranged onto or inside the detection case. The battery may be a rechargeable battery. Alternatively, the detection system may be powered from the battery or batteries related to the portable device. The detection system may be powered from the battery or batteries related to the portable device using power transmitting components for wired or wireless power transmission.

A method for detecting hazardous particles using a detection case comprises the following steps:
detecting at least one of hazardous particles and/or gas,
identifying the hazardous particles and/or gas,
sending a warning signal using a transmitter, such that a user becomes aware of a hazardous situation.

The detection case is configured to receive a portable device belonging to a user. The detection case may fully or partially cover the portable device. The detection case comprises a detection system having a detection system for detecting hazardous particles. The detection case comprising the detection system is capable of warning the user of hazardous situations related to the presence of hazardous particles. The detection system is configured to send a warning signal using a transmitter. The user receives the warning signal, and the user becomes aware of the hazardous situation.

The detection system detects hazardous particles using detection components. The detection system may comprise sensors configured to detect hazardous particles. The term hazardous particles includes every particle, which is a hazard to a person/user, for example: flammable gases, oxygen depletion, and an extensive range of toxic gases, smoke etc.

Alternatively, a method for detecting hazardous particles using a portable device, such as a mobile phone, a tablet, or a laptop comprises detecting a plurality of hazardous particles and/or gases using at least one detection unit, and sending a warning signal using a transmitter, such that a user becomes aware of the hazardous situation.

The control unit may be a computer. The computer may be built in the detection case or the portable device. A computer program product comprises instructions which, when the program is executed by the computer, cause the computer to carry out one or more of: detecting the hazardous particles and/or gases, and sending a warning signal using a transmitter, such that a user becomes aware of the hazardous situation.

The computer program product may further comprise instructions which, when the program is executed by the computer, cause the computer to measure and/or analyze the contents of hazardous particles. The computer program product may further comprise instructions which, when the program is executed by the computer, cause the computer to store the data related to the measuring and/or analyzing of the contents of hazardous particles in a log file.

The computer-readable storage medium may also comprise instructions which, when executed by a computer, causes the computer to store the data related to the measuring and/or analyzing of the contents of hazardous particles in a log file.

In an embodiment, the method comprises further steps of receiving instructions regarding the hazardous particles and/or gas to detect.

A user may use a portable device or an external computer to configure the detection system, such that the settings for the detection system for detecting hazardous particles and/or gases can be initialized or changed. The user may use a user interface to access the detection system or part of the detection system, such as the detection units. The instructions may comprise information regarding specific hazardous particles and/or gases and how to detect the particles and/or gases, as well as instructions regarding levels and hysteresis related to warning signals.

The systems and methods have now been explained with reference to a few embodiments which have only been discussed in order to illustrate the many possibilities and varying design possibilities achievable with the detection case for a portable device or a portable device capable of detecting hazardous particles according to the present disclosure.

### BRIEF DESCRIPTION OF THE DRAWINGS

The embodiments of the systems and methods are described in the following with reference to:

FIG. 1a and FIG. 1b showing a detection case configured to receive a portable device, according to multiple embodiments.

FIG. 2 shows a detection case, according to an embodiment.

FIG. 3 shows a detection cavity, according to an embodiment.

FIG. 4 shows a detection system in a detection cavity, according to an embodiment.

FIG. 5a, FIG. 5b, and FIG. 5c show a detection case and a detection device, according to an embodiment.

FIG. 6 shows a detection case comprising a detection device in an exploded view, according to an embodiment.

FIG. 7 shows a detection device integrated in a mobile device, according to an embodiment.

FIG. 8 illustrates a block diagram of a function related to a detection system, according to an embodiment.

In the explanation of the figures, identical or corresponding elements are provided with the same designations in different figures. Therefore, explanation of all details may not be given in connection with each single figure/embodiment.

### DETAILED DESCRIPTION

Embodiments are explained in the following detailed description. It is to be understood that the systems and methods are not limited in scope to the following description or by the illustrations in the drawings. The systems and methods are capable of other embodiments and of being practiced or carried out in various ways. The figures explain a detection case, but the detection case could be the housing of a mobile phone, a tablet, or a laptop.

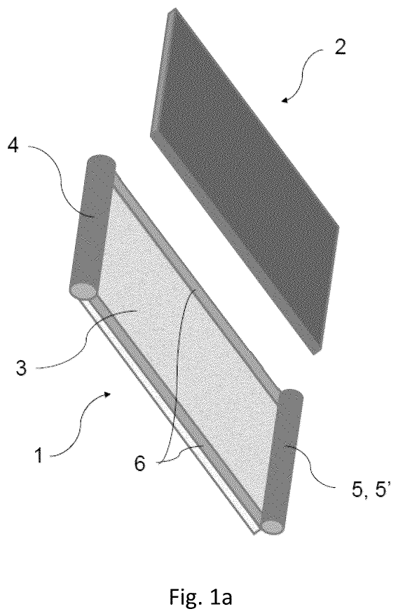

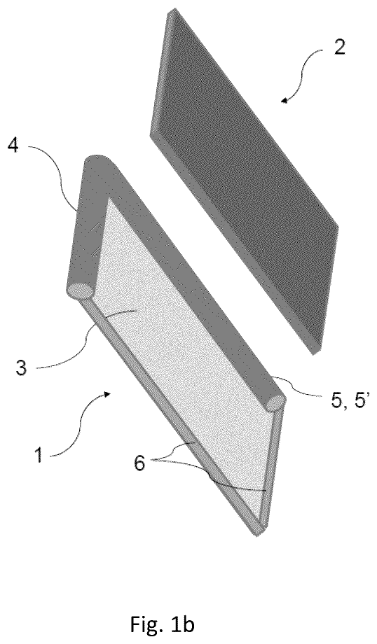

FIG. 1a and FIG. 1b show an embodiment of a detection case 1 configured to receive a portable device 2. The detection case 1 may fully or partially cover the portable device. The portable device may be a mobile phone, tablet, laptop or similar item. The portable device may comprise components for wireless communication.

The detection case 1 comprises two transverse sides opposite each other and two elongated sides opposite each other. The detection case 1 comprises a first detection cavity 4 and second detection cavity 5. The detection case 1 also comprises a back part 3 and supporting sides 6. In these embodiments, the detection case has a longitudinal direction. The portable device 2 may be a mobile phone, tablet, laptop or similar item.

In FIG. 1a the detection cavities 4, 5, are arranged along each of the transverse sides of the detection case 1. The detection cavities 4, 5, may alternatively be arranged along each of the elongated sides of the detection case 1. The first and the second support sides 6, which, in this embodiment, are the elongated sides of the detection case 1, are arranged opposite each other along the longitudinal direction. The supporting sides 6 extend from the first detection cavity 4 to the second detection cavity 5. The back part 3 of the detection case 1 covers most of the backside of the portable device 2, when the portable device 2 is arranged in the detection case 1.

In FIG. 1b the detection cavities 4, are arranged along two transverse sides opposite each other and two elongated sides opposite each other of the detection case 1. The first detection cavity 4 and the second detection cavity 5, are a transverse side and an elongated side of the detection case 1, where the first detection cavity 4 and the second detection cavity 5 are arranged adjacent to each other in one corner of the detection case. The first and the second supporting side 6, are a transverse side and an elongated side of the detection case 1, where the first and the second supporting side 6 are arranged adjacent to each other in another corner of the detection case. The detection case 1 covers most of the backside of the portable device 2, when the portable device 2 is arranged in the detection case 1.

The detection case 1 comprises a first detection cavity 4 and second detection cavity 5. Alternatively, one of the cavities may be a reference cavity 5′.

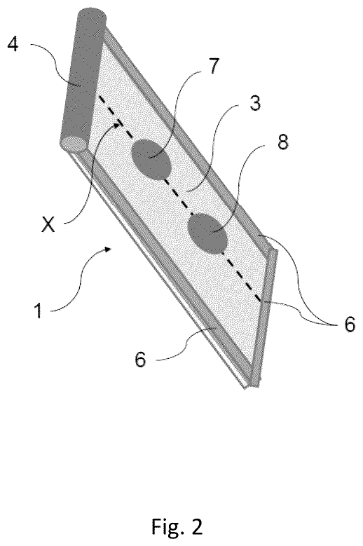

FIG. 2 shows a further embodiment of a detection case 1. The detection case 1 has a longitudinal direction X. The detection cavity 4 may be arranged along one of the transverse sides of the detection case 1. The support sides 6 are arranged along the sides that do not comprise a detection cavity 4.

The detection case 1 comprising the detection system may comprise a control unit 7 for analyzing and processing measurements from the detection system inside the detection cavity. The control unit 7 may be arranged adjacent to the back part 3 of the detection case 1. Alternatively, the control unit 7 may be molded into the back part 3 of the detection case 1. A transmitter 8 may be arranged adjacent to the back part 3 of the detection case 1. Alternatively, the transmitter 8 may be molded into the back part 3 of the detection case 1. The control unit 7 comprises a transmitter 8, where the control unit 7 is configured to send a warning signal using the transmitter 8, for example to send the warning signal to the portable device, when detecting hazardous particles. The transmitter 8 is configured to send the warning signal, via a wireless or wired connection, to the portable device 2.

The detection case 1 comprises a detection cavity 4 and a reference cavity 5′, for example as illustrated in FIG. 1a or FIG. 1b. The detection system may be configured to compare the detected value measured in detection cavity 4 to the referenced value measured in reference cavity 5′. If the detected value of the measurement from detection cavity 4 differs from the reference value of the measurement from the reference cavity 5, there may be hazardous particles in the surroundings, which may cause a hazardous situation for a user. The control unit 7 activates the transmitter 8 which sends a warning signal, such that the user will become aware of the hazard situation.

The transmitter 8 may be a sound emitting unit comprised in the detection case or on the back part 3. The sound emitting unit may, for example, be a mechanical, an electromechanical, or a piezoelectric unit.

Alternatively, the transmitter 8 emits a visual warning signal. The visual warning signal may be emitted from a solid-state lighting material or solid-state lighting component, which changes color when activated by the control unit 7. For example, a solid-state component may be a light emitting diode or similar device. The material surrounding the detection cavity 4 may comprise solid state lighting material, which, when activated, changes color and/or pulses the light emitted from the solid-state lighting material. Alternatively, the material surrounding the detection cavity 4 may comprise transparent material, which can be illuminated using a solid-state lighting component.

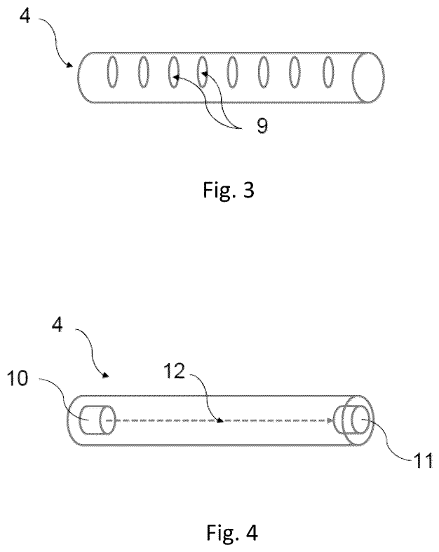

FIG. 3 shows an embodiment of a detection cavity 4. The detection cavity 4 comprises at least one aperture 9. Hazardous particles and/or gases are capable of entering the aperture 9 into the detection cavity 4. The detection cavity 4 comprises detection components for detecting hazardous particles. The detection system is configured to detect the presence of hazardous particles inside the detection cavity 4. If one of the cavities is a reference cavity, the cavity may not comprise any aperture 9.

Alternatively, the detection system comprises a transceiver. The transceiver is configured to send and receive signals to and from the portable device 2. When the detection system detects hazardous particles in the detection cavity 4, the transceiver is configured to send at least one warning signal to the portable device, and if configured so, also to receive a reply signal from the portable device 2.

The detection system may also comprise communication components configured to communicate with a portable device 2. The control unit 7 controls the communication components. The communication components are configured to provide a data communication between the detection system comprised in the detection case 1 and the portable device 2. The detection case 1 may also comprise at least one battery for powering the control unit 7, the transmitter 8, a transceiver, etc. The battery may be a rechargeable battery. The battery may be arranged adjacent to the back part 3 of the detection case 1. Alternatively, the battery may be molded into the back part 3 of the detection case 1.

FIG. 4 shows an embodiment of detection components in a detection cavity 4,5. The detection system is configured to detect hazardous particles and/or gases using the detection components 10,11. For example a smoke detection system, such as a photo-electric smoke detector uses the light scatter principle. The detection components comprise a pulsed infrared light from an LED 10, which pulses a beam of light 12 in or into the detection cavity 4 in predetermined intervals, to check for smoke particles. The beam of light 12 is directed at a light receiving sensor 11. When a fire breaks out, smoke particles will enter the detection cavity 4 through the apertures illustrated in FIG. 3. The smoke enters the detection cavity 4, and the smoke particles cause the infrared light 12 to be scattered onto the light receiving sensor 11. Once the scattered light 12 reaches the light receiving sensor 11, the amount of light can be measured as a detected value. If the detected value crosses a predetermined level, the detection system detects the presence of hazardous particles and/or gases inside the detection cavity 4. A warning signal is then sent to the transmitting unit. The transmitter 8 alerts the user, such that a user becomes aware of the hazardous situation. Other smoke detection systems may be used as an alternative to the above-mentioned smoke detection system.

FIG. 5a, FIG. 5b, and FIG. 5c show a further embodiment of a detection case and a detection device. The detection system 18, as shown in FIG. 5a, comprises in this embodiment an elongated PCB 20. The PCB 20 comprises a first detection unit 21 and a second detection unit 22. The first detection unit 21 and the second detection unit 22 are multi detection sensors capable of detecting a plurality of hazardous particles and/or gases. The first detection unit 21 is arranged in a first end of the PCB 20. The second detection unit 22 is arranged in a second end of the PCB 20, where the second end is opposite the first end. A wireless charger 23 is arranged relative to the PCB 20. The wireless charger 23 is, in this embodiment, a coil. A power storage unit 24 is arranged relative to the PCB 20 and the wireless charger 23. The power storage unit 24 is a rechargeable battery. The detection system 18 is configured to charge the power storage unit 24 using the wireless charger 23. The detection case has a limited thickness. The detection system's electronic components and PCB are arranged such that the detection system's electronic components and PCB are distributed evenly along the inner surface of the detection case. The length L and width W of the detection system may be less than the detection case volume for housing a mobile device. The height H of the PCB may define the distance between the inner surface and the protection plate. Alternatively, the height H of the PCB may define the distance between the inner surface and a back side of mobile device. The distance between the inner surface and the back side of mobile device is related to the distance when the mobile device is resting in the protection case.

The detection system 18 comprises a wireless communication unit 19. The wireless communication unit 19 is configured to communicated with a portable device. The wireless communication unit may also be configured to be in data communication with devices other than the portable device. The wireless communication unit 19 is arranged relative to the PCB 20.

The detection case 1 is, in this embodiment, divided into two case parts. The case parts are a case front part 14 and a case back part 15. A portable device 2 is arranged inside the case front part 14. The case front part 14 may, in use, be capable of being releasably fastened to the case back part 15, enclosing the detection system 18. The case back part 15 comprises apertures 9′,9″ which provide a fluid communication between the respective detection units 21, 22. A recess is provided from an edge of the case back part to each of the apertures 9′,9″ for conducting the ambient air or liquid towards the respective detection units 21, 22. The recess may also conduct liquid, such that the liquid may be tested for hazardous particles. Alternative solutions may be provided for conducting the ambient air or liquid towards the respective detection units 21, 22, which are not shown in this embodiment.

A protrusion member 28 is provided in each corner of the outer surface 27, wherein protrusion member 28 extends away from the outer surface 27. The protrusion members 28 provide space between the outer surface 27 and the resting surface, such that the detection case 1 is capable of resting elevated on a resting surface, e.g. a table or the like, and capable of conducting the ambient air towards the respective detection units 21, 22.

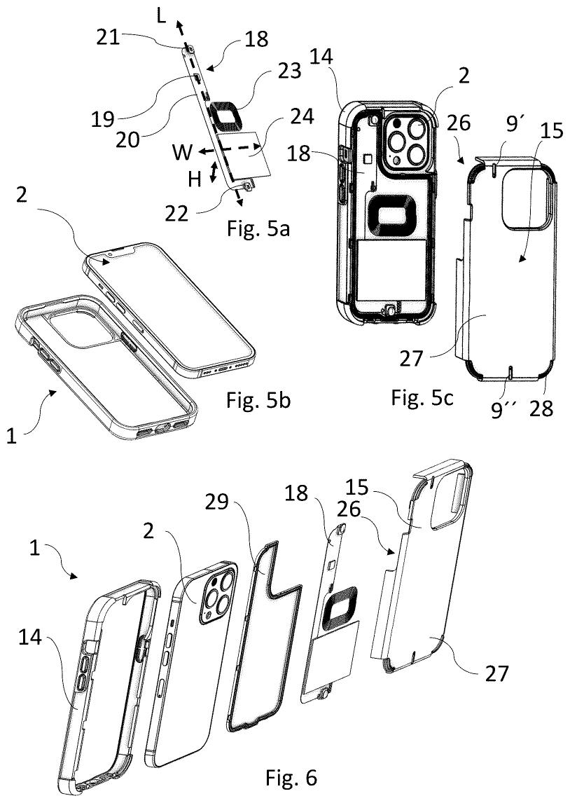

FIG. 6 shows a further embodiment of a detection case 1 comprising a detection device in an exploded view. The detection case 1 may comprise a case front part 14 having an opening where a mobile device 2 may be arranged. The case front part 14 may be arranged along the edge of the mobile device 2. The detection case 1 may comprise a protection plate 29. The protection plate 29 is arranged between the back side of the mobile device 2 and the detection system 18, such that the detection system's electronic components are protected. The detection system 18 is arranged between the inner surface of the case back part 15 and the protection plate 29. The detection system may be molded or partly molded into the detection case 1, such that the protection plate 29 and case back part 15 enclose detection system 18.

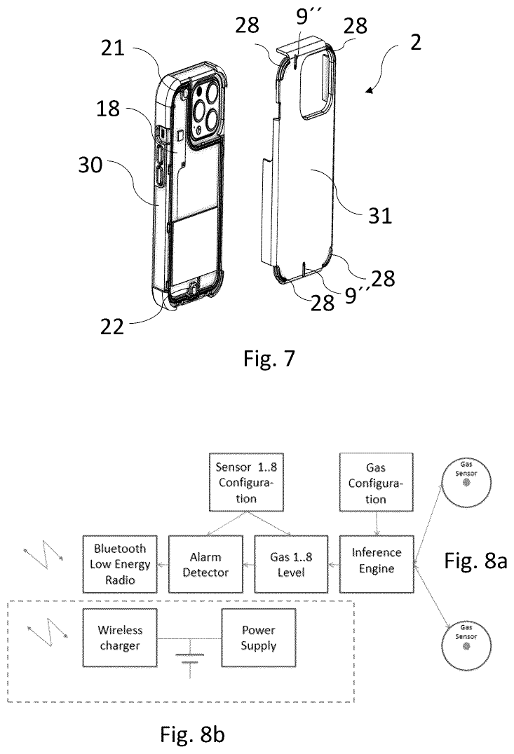

FIG. 7 shows a detection system 18 integrated in a mobile device 2. The housing of the mobile device is divided into two parts, a front part 30 and a back part 31. The detection system 18 is arranged on the back side of the front part 30, such that the detection system is adjacent to the back part 31 of the housing when in use. The detection system 18 may be powered from the mobile device power storage unit or the detection system's power storage unit may be recharged by the mobile device's charging unit. The back part 31 comprises apertures 9′,9″ which provide an air communication between the respective detection units 21, 22. A recess is provided from an edge of the case back part to each of the apertures 9′,9″ for conducting the ambient air towards the respective detection units 21, 22. A protrusion member 28 is provided in each corner of the outer surface 27, wherein protrusion member 28 extends away from the outer surface 27. The protrusion members 28 provide space between the outer surface 27 and a resting surface, such that the detection case 1 is capable of resting elevated on a resting surface, e.g. a table or the like, and capable of conducting the ambient air towards the respective detection units 21, 22.

FIG. 8a and FIG. 8b illustrate a block diagram of a function related to a detection system comprising a power system, wherein FIG. 8a refers to the main detection system and FIG. 8b refers to the power system. The one or more multi detection sensors are capable of detecting a plurality of hazardous particles and/or gases. The multi detection sensor may be a Bosch BME688 gas sensor with Artificial Intelligence or a similar detector, but is not limited to such sensor technology.

The gas configuration comprises information regarding the hazardous particles and/or gases specific for the hazardous particles and/or gases to be detected. The information regarding the gas configuration is correlated with the hazardous particles and/or gases detected by the gas sensors, using an inference engine. The inference engine may be based on an ML or AI trained engine.

The sensor configuration comprises predetermined information regarding detection levels related to each of the hazardous particles and/or gases which can be detected by the system. When detected hazardous particles and/or gases are identified, their levels are determined. Based on the information, an alarm detector sets an alarm if the level is exceeded, otherwise not. The alarm may be sent to the mobile device and/or to a buzzer, such that the user will become aware of the hazardous situation.

FIG. 8b illustrates that the power system is a wireless power system capable of being charged by a wireless power supply. The power system is capable of being charged from the same wireless power supply as the mobile device. The power system is capable of being charged at the same time as the mobile device is being charged.

What is claimed is:

1. A detection case configured to receive a portable device, the detection case comprising:
a detection system configured to detect hazardous particles and/or gases, wherein the detection system is arranged adjacent an inner surface of the detection case, wherein the detection system comprises a detection unit trained to detect the hazardous particles and/or gases using machine learning or artificial intelligence features;
wherein the detection unit includes a gas scanner function customized with respect to sensitivity; and
the detection unit and/or the detection system is trained to a specific customized application using the machine learning or the artificial intelligence features.

2. The detection case according to claim 1, wherein the detection system is built into a housing of the portable device comprising communication components configured to communicate with other portable devices.

3. The detection case according to claim 1, wherein the detection case comprises a protection plate for protecting the detection system, wherein the detection system is arranged between a back part of the detection case and the protection plate, wherein the detection system comprises a PCB, wherein the PCB comprises a first detection unit that is arranged in a first end of the PCB, and a second detection unit that is arranged in a second end of the PCB, wherein the second end is opposite to the first end.

4. The detection case according to claim 1, wherein the detection case comprises a detection unit and a reference unit, wherein the detection system is capable of comparing a measured detected value from the detection unit to a measured reference value from the reference unit.

5. The detection case according to claim 1, wherein the detection case comprises at least one aperture, wherein at least one detection unit is arranged adjacent to the inner surface of the detection case relative to said aperture, wherein said aperture is configured to provide fluid communication between ambient surroundings and the detection unit.

6. The detection case according to claim 1, wherein the detection case comprises at least one protrusion member on an outer surface, such that when the detection case is arranged on a resting surface with the outer surface facing the resting surface, the protrusion member provides space between the outer surface and the resting surface.

7. The detection case according to claim 1, wherein the detection system comprises a power storage unit, wherein the power storage unit is configured to receive power from a charging unit.

8. The detection case according to claim 7, wherein the charging unit comprises a wireless charger configured to receive wireless charging, wherein the detection system is configured to be wirelessly charged at a same time as the portable device when using a wireless charging phone case.

9. The detection case according to claim 8, wherein the wireless charger comprises a coil.

10. The detection case according to claim 1, wherein the detection system comprises a transmitter configured to send at least one warning signal such that when the detection system detects the hazardous particles and/or gases the detection system transmits at least one warning signal.

11. The detection case according to claim 1, wherein the detection system comprises a processor for processing at least one detected hazardous particle and/or gas, wherein the detection system is configured to identify the at least one detected hazardous particle and/or gas and/or a concentration level of the hazardous particle and/or gas.

12. The detection case according to claim 1, wherein the detection system comprises a processor for processing a plurality of detected hazardous particles and/or gases simultaneously, wherein the detection system is configured to identify the plurality of detected hazardous particles and/or gases and/or a concentration level of the hazardous particles and/or gases.

13. The detection case according to claim 1, wherein the detection system is in data communication with the portable device and receives instructions from the portable device to detect predetermined hazardous particles and/or gases.

14. The detection case according to claim 1, wherein the detection case comprises one or more of:
a breathalyzer;
a date rape drug tester; and
a smoke/fire detector.

15. The detection case according to claim 1, wherein the detection system comprises predetermined information regarding detection levels related to each of the hazardous particles and/or gases that can be detected by the detection system.

16. The detection case according to claim 1, wherein the detection unit comprises pulsed infrared light from an LED that pulses a beam of light in or into a detection cavity at predetermined intervals to check for smoke particles, wherein the beam of light is directed at a light receiving sensor.

17. A method for detecting hazardous particles and/or gases using a detection case according to claim 1, comprising:
training the detection unit to detect the predetermined hazardous particles and/or gases using machine learning or artificial intelligence;
detecting at least one of the hazardous particles and/or gases;
identifying the hazardous particles and/or gases; and
sending a warning signal using a transmitter.

18. The method according to claim 17, further comprising receiving instructions regarding the hazardous particles and/or gas to detect.
